(19) United States
(12) Patent Application Publication
(10) Pub. No.: US 2003/0212014 A1
(43) Pub. Date: Nov. 13, 2003

(54) Methods fo treating conditions associated with insulin resistance with aicar, (5-amino-4-imidazole carboxamide riboside) and related compounds

(76) Inventors: Neil Ruderman, Newton, MA (US); Edward W Kraegen, Sydney (AU); Yasuo Ido, Brookline, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/343,399

(22) PCT Filed: Jul. 31, 2001
(86) PCT No.: PCT/US01/24194

(30) Foreign Application Priority Data
Aug. 9, 2000 (US) 00/40607
Jun. 6, 2001 (US) 01/18467

(51) Int. Cl. A61K 31/7056

(52) U.S. Cl. 514/43

(57) ABSTRACT

The long-term usage of AICR (5-aminio, 4-imidazole carboxamide riboside) to produce sustained metabolic and biological changes in mammals that overcome insulin resistance, i.e., increase insulin sensitivity, and result in benefits in diseases and conditions such as diabetes, hypertension, atherosclerosis, polycystic ovary syndrome and gallstones is described long-term usage of AICAR, particularly intermittent administration, e.g., three days per week, appears to have some of the positive effects of exercise, having an impact on the amount Of food consumed by a subject and resulting in reduced fat build-up and increase in muscle mass. Therefore, AICAR administration has a positive impact in reducing obesity. AICAR can also Prove useful in preventing or treating vascular diseases associated with hyperglycemia, high plasma levels of free fatty acids (FFA) and triglyceride, and insulin resistance by virtue of the fact that this agent activates fatty acid oxidation. Animal tests have Shown that chronic intermittent treatment with AICAR has not resulted in any noticeable toxic effects. AICAR and related compounds are activators of AMP-activated protein kinase (AMPK) and, furthermore, are effective at decreasing malonyl CoA levels in the animal.

### CROSS REFERENCE TO RELATED APPLICATIONS

This application claims the priority of the following applications: U.S. Provisional Application No. 60/222,131, filed Jul. 31, 2000 entitled, USE OF AICAR (5-AMINO-4-IMIDAZOLE CARBOXAMIDE RIBOSIDE) AND RELATED COMPOUNDS TO TREAT INSULIN RESISTANCE; International Application No. PCT/US00/40607, filed Aug. 9, 2000 entitled, METHOD OF MAINTAINING VASCULAR INTEGRITY USING AICAR (5-AMINO-4-IMIDAZOLE CARBOXAMIDE RIBOSIDE) AND RELATED COMPOUNDS; and International Application No. PCT/US01/18467 filed Jun. 6, 2001 entitled, USE OF AICAR (5-AMINO-4-IMIDAZOLE CARBOXAMIDE RIBOSIDE) AND RELATED COMPOUNDS FOR THE PREVENTION AND TREATMENT OF OBESITY, the whole of which are hereby incorporated by reference herein.

### STATEMENT REGARDING FEDERALLY SPONSORED RESEARCH OR DEVELOPMENT

This invention was made in part with United States Government support under Contract Number NK 19514 and Grant Number HL-55854-05, both awarded by the National Institutes of Health. Therefore, the U.S. Government has certain rights in the invention.

### BACKGROUND OF THE INVENTION

AMP-activated protein kinase (AMPK) is a cytoplasmic enzyme that has been shown to exist in both the liver and skeletal muscle. As its name indicates, AMPK is activated by increasing levels of AMP and, secondarily, by an increase in the ratio of AMP to ATP in the cell. AMP levels rise in the cell as ATP is hydrolyzed to ADP and Pi. Two molecules of ADP, through the action of myokinase, also known as adenylate kinase, produce one molecule of ATP and one molecule of AMP. In addition to its activation by AMP, AMPK is activated through phosphorylation by an upstream kinase called AMPK kinase (AMPKK). AMP also allosterically activates AMPKK. Phosphorylation of AMPK by AMPKK makes it a poor substrate for phosphatases. All these factors combined together make AMPK very sensitive to minimal fluctuations in cellular AMP levels.

AMPK has several known substrates, specifically enzymes that it can phosphorylate and modulate. In the liver, AMPK has been shown to phosphorylate hydroxymethyl glutaryl CoA (HMGCOA) reductase and acetyl CoA carboxylase (ACC), inhibiting the actions of both enzymes. Reducing HMGCOA reductase activity inhibits cholesterol synthesis, and reducing ACC activity decreases the generation of malonyl CoA, an intermediate in fatty acid synthesis. In skeletal muscle, AMPK also is an inhibitor of carnitine palmitoyl transferase I, which regulates the uptake of fatty acids into mitochondria where they are oxidized. In addition, AMPK has been shown to increase glucose transport into the muscle.

To stimulate AMPK experimentally, a compound called AICAR (5-aminoimidizole-4-carboxamide riboside) is used. AICAR (also known as acadesine) is a naturally occurring analogue of adenosine that is taken up by muscle and liver and phosphorylated to form 5-aminoimidazole-4-carboxamide-1-β-D-ribofuranosyl-5′-monophosphate (ZMP), which activates both AMPK and AMPKK. In turn, AMPKK phosphorylates AMPK and activates it even further.

Various studies have been carried out to evaluate the effects of short-term usage of AICAR in experimental situations. U.S. Pat. No. 5,443,836 discloses that short-term usage has resulted in an increase in the local concentration of adenosine, which may benefit patients with a wide variety of disorders associated with decreased blood flow (ischemia). These disorders include stroke, heart attack and adverse effects associated with ischemia of the liver, bowel and, by inference, other organs. All of the studies relative to this use of the agent were performed in humans or experimental animals for relatively short periods of time (usually less than 80 hrs).

U.S. Pat. No. 5,658,889 discloses that the short-term usage of AICAR in very high doses (500 mg/kg/twice daily) lowers blood glucose levels in control and diabetic rats. This patent also discloses studies in which diabetic rats were treated with such a regimen for 23 days with an apparent decrease in the severity of the diabetes as judged by lower blood glucose levels and decreased polyuria (i.e., a decrease in the large urine volume).

### BRIEF SUMMARY OF THE INVENTION

We have found that long-term usage of AICAR (5-amino, 4-imidazole carboxamide riboside) produces sustained metabolic and biological changes in mammals that overcome insulin resistance, i.e., increase insulin sensitivity, and can result in benefits in diseases and conditions such as diabetes, hypertension, atherosclerosis, polycystic ovary syndrome and gallstones. In addition, long-term usage of AICAR, particularly intermittent administration, e.g., three days per week, appears to have some of the positive effects of exercise, having an impact on the amount of food consumed by a subject and resulting in reduced fat build-up and increase in muscle mass. Therefore, AICAR administration has a positive impact in reducing obesity. AICAR can also prove useful in preventing or treating vascular diseases associated with hyperglycemia, high plasma levels of free fatty acids (FFA) and triglyceride, and insulin resistance by virtue of the fact that this agent activates fatty acid oxidation. Animal tests have shown that chronic intermittent treatment with AICAR has not resulted in any noticeable toxic effects. AICAR and related compounds are activators of AMP-activated protein kinase (AMPK) and, furthermore, are effective at decreasing malonyl CoA levels in the animal.

Thus, in general, the method of the invention is directed to the use in a patient of low dose, sustained and, preferably, intermittent administration of activators of AMP-activated protein kinase (AMPK), most preferably AICAR (5-amino-4-imidazole carboxamide riboside) and related compounds, that, furthermore, are effective at decreasing malonyl CoA levels, for prophylaxis or treatment of a disease or condition associated with hyperglycemia, insulin resistance or obesity, commonly referred to as the insulin resistance syndrome or syndrome X. Other compounds useful in the method of the invention include analogs of AICAR (such as those disclosed in U.S. Pat. No. 5,777,100, hereby incorporated by reference herein) and prodrugs or precursors of AICAR (such as those disclosed in U.S. Pat. No. 5,082,829, hereby incorporated by reference herein), which increase the bioavailability of AICAR, all of which are well-known to those of ordinary skill in the art.

In one aspect, the disease or condition is vascular disease associated with metabolic abnormalities, in particular atherosclerotic vascular disease. In another aspect, the invention is particularly directed to a method for prophylaxis or treatment of obesity that includes providing a patient, particularly a human patient, suffering from or believed to be at risk of suffering from obesity and administering intermittently to the patient a therapeutic composition including an amount of AICAR, AICAR analog or AICAR precursor that is therapeutically effective at preventing or treating obesity by reducing abdominal fat in the patient.

Preferably, the frequency of administration of the therapeutic composition according to the method of the invention ranges from once per week to every other day. Furthermore, the preferred route of administration is by subcutaneous injection or oral ingestion. AICAR administration according to the method of the invention is effective at reducing abdominal fat, particularly intra-abdominal fat, without acute side effects, e.g., hypoglycemia (low glucose levels) or hyperlacticacidemia (high lactic acid levels).

It is understood that those with skill in this and related fields (e.g., synthetic organic chemistry) will identify other agents that increase the oxidation of fatty acids and decrease its esterification (or decrease levels of long chain fatty acyl CoA in the cytosol of the cell) and which might function equally as well as AICAR, i.e., agents that specifically are activators of AMPK and, furthermore, are effective at decreasing malonyl CoA levels in a patient. Because of the low absorption rate of the AICAR when administered orally (approximately 5%), compounds which release AICAR after ingestion, methods that will result in the release of AICAR (e.g., encapsulation), and other methods of administration (e.g., usage of pumps that deliver small amounts continuously) will need to be explored. In addition, those with skill in the field will identify variations of the invention which are consistent with the disclosure herein.

### BRIEF DESCRIPTION OF THE DRAWINGS

Other features and advantages of the invention will be apparent from the following description of the preferred embodiments thereof and from the claims, taken in conjunction with the accompanying drawings, in which:

FIG. 1 shows the metabolic effects of AICAR and inhibition of apoptosis;

FIGS. 2A-2B show AMP-activated protein kinase activity;

FIG. 3 shows Acetyl Co-A Carboxylase activity;

FIG. 4, Table 1, shows the effects of AICAR incubation on metabolism after 2 hours with no carnitine;

FIG. 5 shows fatty acid oxidation in the presence and absence of AICAR;

FIG. 6, Table 1A, shows ATP generation in HUVEC over 2 hours, with carnitine and prelabeling, including data for the second hour;

FIGS. 7A-7C show the effects of AICAR on HUVEC metabolism after 16 hours incubation with 30 mM glucose;

FIG. 8 shows the effects of 2-bromopalmitate and high levels of free fatty acid on apoptosis in HUVEC;

FIGS. 9A-9B show the effects of AICAR on hyperglycemia-induced apoptosis. (A) Typical TUNEL staining (dark brown) with methylene blue counter-staining. TUNEL positive cells show a typical apoptotic configuration with shrinkage and nuclear fragmentation. Representative pictures from cells incubated for 72 hr with 5 or 30 mM glucose or 30 mM glucose +1 mM AICAR are shown. (B) HUVECs were incubated with Medium 199 +10% FBS ±1 mM AICAR as indicated. The percentage of TUNEL positive cells was determined in 3 wells of 6 well plates (24 hr) or in eight of 60 mm dishes. Data are means ±SD.*p<0.05; vs 5 mM glucose. † p<0.05; vs 30 mM glucose alone;

FIG. 10 shows the effects of AICAR on fatty acid metabolism, AMP-kinase and malonyl-CoA levels. All parameters were measured in HUVECs incubated with Medium 199 +10% FBS±1 mM AICAR for 24 hr. Fatty acid oxidation was measured over 2 hours after 24 hr prelabeling with ³H-palmitate. Data are means ±SD (n=4−6). *P<0.05; vs 5 mM glucose. † p<0.05; vs 30 mM glucose;

FIGS. 11A-11B show (A) representative blots showing effects of 24 h incubation with 5 or 30 mM glucose ±1 mm AICAR on Akt phosphorylation and abundance and (B) Akt phosphorylation/unit abundance. Results are means±SE (n=4) *p<0.05 vs 5 mM glucose. † p<0.05 vs 30 mM glucose;

FIG. 12, Table 2 shows the effect of 25 days of treatment with AICAR (250 mg/kg) on fat depot weight and muscle triglyceride;

FIG. 13, Table 3 shows the effect of 25 days of treatment with AICAR (250 mg/kg) on body and organ weight;

FIG. 14, Table 4, shows the acute effects of AICAR on plasma metabolites and hormones;

FIG. 15, Table 5 shows the effect of 25 days of treatment with AICAR (250 mg/kg) on plasma metabolites and hormones;

FIG. 16A, Table 6A shows the effect of 16 weeks of treatment with AICAR (250 mg/kg) on adipose depot weight;

FIG. 16B, Table 6B shows the effect of 16 weeks of treatment with AICAR on body and organ weight;

FIG. 17, Table 7 shows the effect of 16 weeks of treatment with AICAR (250 mg/kg) on weight, blood and plasma;

FIG. 18, Table 8 shows food consumption and body weight over 12 weeks in control and AICAR treated rats;FIG. 19 shows the effect of AICAR administration (3 times/week) on food intake in rats on chow diet;

FIG. 20 shows blood glucose levels following AICAR injection (250 mg/kg);

FIG. 21 shows body weight changes in rats injected with AICAR (250 mg/kg) 3 times/week;

FIG. 22 shows the acute effects of AICAR administration on gastroc. muscle malonyl CoA and ACC activity;

FIG. 23 shows the acute effects of AICAR administration on liver malonyl CoA and ACC activity;

FIG. 24 shows plasma glucose levels as a function of time as determined in an oral glucose tolerance test on rats treated with AICAR;

FIG. 25 shows plasma insulin levels as a function of time as determined in an oral glucose tolerance test on rats treated with AICAR;

FIG. 26 shows plasme parameters after AICAR injection;

FIG. 27, Table 9, shows tissue glycogen and triglyceride content;

FIG. 28 shows parameters of insulin sensitivity determined during the clamp and with the disappearance of tracers from plasma;

FIG. 29, Table 10, shows plasme parameters at basal state and during insulin stimulation;

FIG. 30 shows glycogen content and insulin stimulated glucose incorporation into glycogen;

FIG. 31, Table 11, shows muscle glycogen, triglyceride and total LCAC content after clamp; and

FIG. 32 shows triglycerides and malonyl CoA content in liver.

### DETAILED DESCRIPTION OF THE INVENTION

Acetyl CoA carboxylase (ACC) is the enzyme responsible for the first committed step in fatty acid synthesis, the carboxylation of acetyl CoA in the cytosol of a cell to produce malonyl CoA. Malonyl CoA is both an intermediate of fatty acid synthesis and an inhibitor of carnitine palmitoyl transferase I (CPTI), the enzyme that regulates the uptake of long chain fatty acid CoA (LCFA CoA) into the mitochondria where these compounds undergo oxidation. Increasing the fuel supply of muscles by treating them with glucose and insulin increases the concentration of malonyl CoA and diminishes fatty acid oxidation by increasing the cellular concentration of citrate, an activator of ACC. Conversely, exercise lowers the concentration of malonyl CoA, not by lowering the concentration of citrate but by activating AMPK, which phosphorylates and inhibits ACC. This effect of AMPK on ACC and fat oxidation dominates even when cellular citrate levels are increased.

By responding to an increase in total AMP levels, which parallels a need for energy, AMPK activation, and the concomitant inhibition of ACC and reduction in malonyl CoA levels, allows the cell to switch from storing fatty acids as triglyceride (i.e., esterified lipid) to oxidizing fatty acids to provide free energy for the biological needs of the organism. In muscle, administration of AICAR (5-amino, 4-imidazole carboxamide riboside), a naturally occurring activator of AMPK, has been shown to reproduce many of the effects of exercise, including phosphorylation and inhibition of ACC, and to increase fatty acid oxidation and glucose transport. In liver, provision of free energy appears to be accomplished by decreasing the synthesis of cholesterol and fatty acids (i.e., by decreasing energy use) as well as by allowing fatty acid oxidation to occur at a higher rate and thus provide more ATP. In support of this view, it has recently been observed that malonyl CoA in certain key tissues (such as heart and skeletal muscle) is a crucial regulator of fat metabolism and energy balance (Abu-Elheiga et al., Science 291:2613-2616, 2001).

Thus, the method of the invention is directed to the use of low dose, chronic (indefinite time) and, preferably, intermittent administration of AICAR (5-amino-4-imidazole carboxamide riboside) and related compounds in the prevention or treatment of obesity, hyperglycemia and closely related disorders, e.g., those associated with insulin resistance. The studies reported herein have been conducted in human umbilical vein endothelial cells (HUVEC) or in juvenile rat models for insulin resistance. We show that AMPK is indeed present in endothelial cells and that its activity is enhanced by the administration of AICAR. In addition, we demonstrate the presence of ACC in these cells and the modulation of its activity and the decrease in malonyl CoA with the rise of AMPK. Furthermore, the provision of AICAR, by activating AMPK, protects HUVEC against both the development of insulin resistance and programmed cell death (apoptosis), both of which typically occur when such cells are incubated at an elevated glucose concentration for 1-3 days. Likewise, it prevents apoptosis in cells incubated with a moderately high concentration of free fatty acids (FFA) e.g., palmitate, such as occurs in patients with diabetes and other disorders associated with insulin resistance.

Based on these observations, we believe that treatment with AICAR, or a derivative thereof or a related compound, will be useful in particular in patients with insulin resistance or diabetes mellitus, especially non-insulin dependent diabetes mellitus (NIDDM). Treatment with AICAR should diminish damage to the endothelium caused by hyperglycemia and free fatty acids, and for this reason it should be useful in preventing and treating various types of vascular disease associated with metabolic abnormalities, including atherosclerotic vascular disease and, in particular, atherosclerosis associated with diabetes and insulin resistance. By virtue of its effects on glucose and fatty acid metabolism in pericytes, this treatment should also be useful in treating and preventing the microvascular complications of diabetes (such as blindness, retinopathy and possibly nephropathy). Furthermore, AICAR should also prove a useful tool as a chronically administered therapeutic agent in a wide array of situations in which endothelial cell integrity is compromised by stress, e.g., hyperglycemia, high plasma free fatty acid levels and to the extent they are caused by alterations in glucose or fatty acid metabolism possibly ischemia and inflammation.

AICAR is poorly absorbed from the gastrointestinal tract (ca. 5%); therefore, it has been administered parentally in vivo. Thus, also described herein are methods that can easily be carried out by procedures well known to skilled chemical pharmacists to develop derivatives of AICAR or other AMPK activators that are better absorbed.

While not being bound by any theory, it is believed that the mechanism of action of AICAR is the following. By activating AMPK, AICAR inhibits acetyl CoA carboxylase (ACC). Inhibition of ACC in turn results in a decrease in the concentration of malonyl CoA, an inhibitor of carnitine palmitoyl transferase I, an enzyme that controls fatty acid oxidation by regulating the transfer of long chain fatty acyl CoA (LCFA CoA) into mitochondria. We have shown in cultured HUVEC that treatment with AICAR activates fatty acid oxidation, diminishes fatty acid esterification and inhibits the programmed cell death (apoptosis) caused by high concentrations of glucose and/or fatty acids. We have also shown that under conditions of high glucose and/or fatty acid concentration, the ability of insulin to activate the enzyme Akt/PKB, which by itself inhibits apoptosis, is depressed. In addition, we have demonstrated that AICAR overcomes this abnormality in insulin action.

We have demonstrated the presence of both AMPK and ACC in the vasculature. We show that AICAR increases AMPK, leading to a decrease in ACC and thus an increase in fatty acid oxidation in the vasculature, as seen in both liver and skeletal muscle. In addition, we show a decrease in glycolysis and increase in glucose and fatty acid oxidation and possibly total levels of ATP. This increase or maintenance of cellular ATP was not associated with a decrease in protein synthesis or Na⁺/K₊pump activity, although it was shown to decrease flux through an as yet unidentified K⁺channel.

The major effect of AICAR on ATP maintenance appeared to be due to a large increase in fatty acid oxidation. Indeed, we found that nearly 40% of the ATP generated by HUVEC incubated for 2 hrs in 6 mM glucose with AICAR was accounted for by fatty acid oxidation (with 60% of this production occurring during the second hour). Furthermore, when glucose was omitted from the medium, the cells were able to maintain their ATP content for at least 2 hrs, despite the fact that nearly all of the ATP they generated was probably the result of fatty acid oxidation.

Our experiments have indicated that AICAR can be administered for upwards of 3 months without obvious toxicity. Our preliminary studies indicate that rodents can tolerate AICAR at a dose of 250 mg/kg administered subcutaneously for upwards of 3 months without evidence of gross toxicity, suggesting it can be used chronically. Others have found numerous toxic side effects at higher doses.

The data show that AICAR substantially diminishes intra-abdominal fat without diminishing the mass of other organs; indeed, if anything, our preliminary data suggest it increases muscle mass. In addition, chronic treatment with AICAR diminished plasma leptin and insulin levels in keeping with decreases in adiposity, and it decreased plasma triglycerides and possibly cholesterol. All of these findings suggest that AICAR chronically increases insulin sensitivity and decreases adiposity and plasma lipids—all of which should decrease a predisposition to atherosclerosis. The improvement in insulin sensitivity should also decrease the risk of other diseases associated with the insulin resistance syndrome (e.g., diabetes, hypertension, gallstones) in humans.

The therapeutic compositions may be administered orally, topically (e.g., by skin patch), or parenterally (e.g., intranasally, subcutaneously, intramuscularly, intravenously, or intra-arterially) by routine methods in pharmaceutically acceptable inert carrier substances. For example, the therapeutic compositions of the invention may be administered according to the method of the invention in a sustained release formulation using a biodegradable biocompatible polymer, or by on-site delivery using micelles, gels or liposomes. The therapeutic compound can be administered to a mammal in a dosage of, e.g., 5 mg/kg/day to 100 mg/kg/day. The dosage levels used in rats in the experiments reported herein correspond to approximately 50 mg/kg human body weight/day or about a 3-4 gram dosage per human/day. Optimal dosage and modes of administration can readily be determined by conventional protocols.

For prophylactic or therapeutic use with human patients, subcutaneous injection of AICAR is the preferred route of administration for long-term treatment, since it should produce a more sustained increase in AICAR concentration in plasma than intraperitoneal administration. Other forms of administration can be developed as described herein to take advantage of forms of AICAR, or of compounds with similar activity, with increased bioavailability. In a human patient, significant reductions in abdominal fat can be determined easily by serial measurement of waist circumference.

It is understood that those with skill in this and related fields (e.g., synthetic organic chemistry) will be able to use methods described herein to develop and identify other agents, e.g., analogs of AICAR and compounds with similar activity, that will function equally well in the method of the invention. Because of the low absorption rate of AICAR when administered orally (approximately 5%), compounds that release AICAR after ingestion, prodrugs that activate the upstream AMPK kinase, methods that will result in the release of AICAR (e.g., encapsulation), and other methods of administration (e.g., usage of pumps that deliver small amounts continuously) will also prove useful. In addition, those with skill in the field will identify variations of the invention which are consistent with the disclosure herein.

The following examples are intended to further illustrate, but not limit, the invention.

### EXAMPLE 1

### Studies with Cultured HUVEC

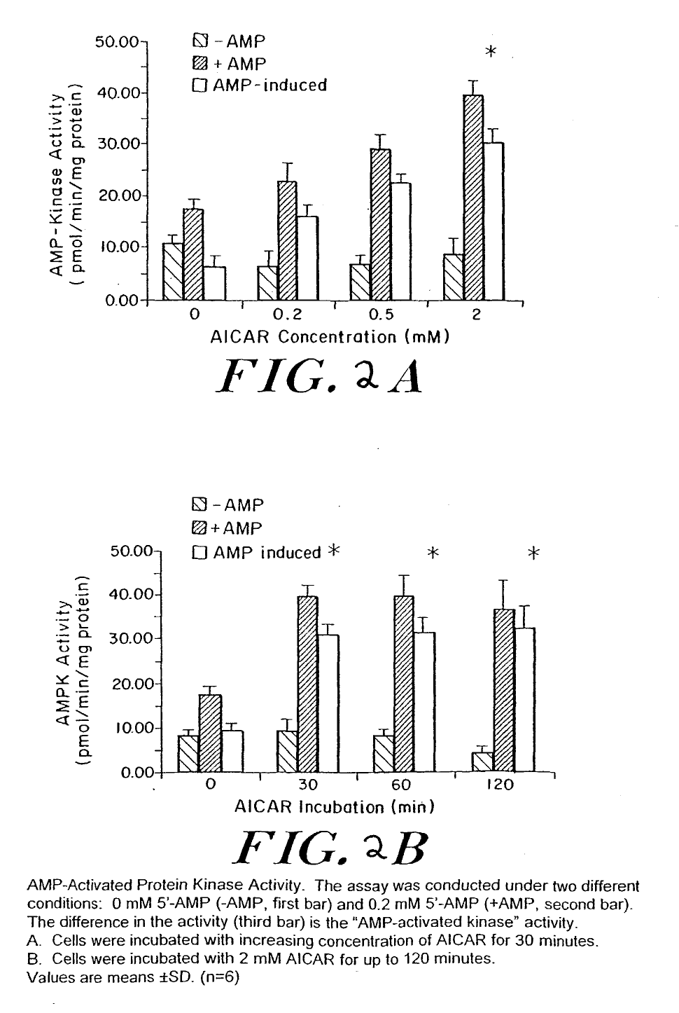

In order to demonstrate the presence of AMP-dependent protein kinase in HUVEC, confluent cells were incubated with varying concentrations of AICAR for 30 minutes (FIG. 2A). The enzyme was assayed in a reaction mixture containing either no AMP or 0.2 mM 5′-AMP. The difference in the activity is the AMP-activated kinase activity. The kinase activity increased from 6.7±0.2 pmol/min/mg protein at 0 mM AICAR incubation, to 15.8±0.4 at 0.2 mM (P=0.05), 22.5±0.3 at 0.5 mM (P=0.05), and 30.9±0.2 at 2 mM AICAR incubation (P=0.002). With increasing AICAR concentration, there was a significant increase in AMPK activity. This indicates the presence of AMPK and AMPKK in HUVEC. Incubation of cells with 2 mM AICAR showed an increase in AMPK activity as compared to control. The change was seen at 30 minutes and persisted for at least 120 minutes (FIG. 2B). The kinase activity increased from 7.3±1.8 at 0 minutes to 30.8 at 30 minutes (P=0.01), 31.5 at 60 minutes (P=0.01) and 32.3 pmol/min/mg protein at 120 minutes (P=0.01).

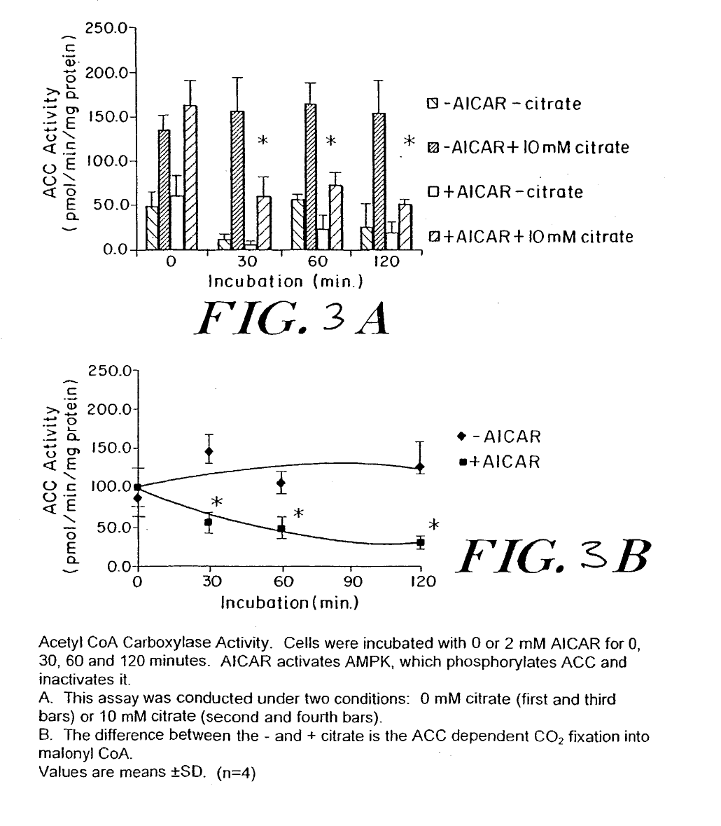

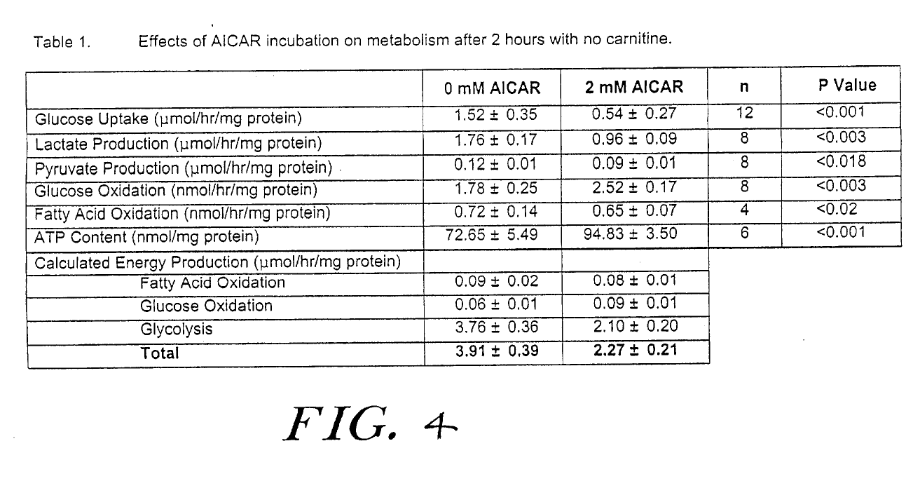

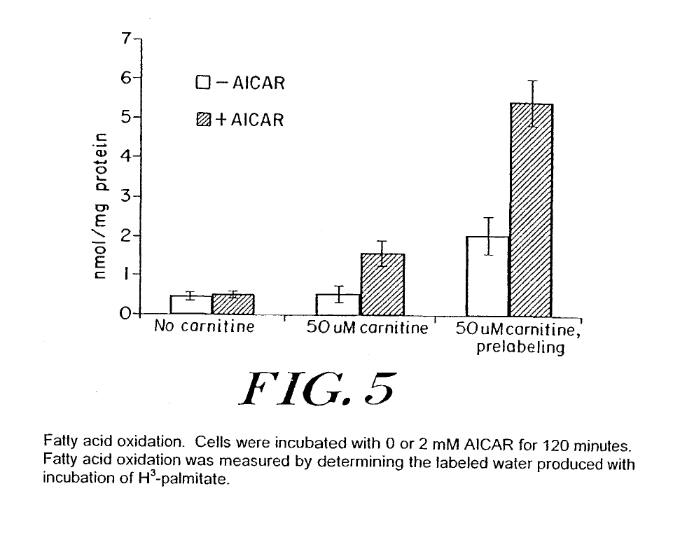

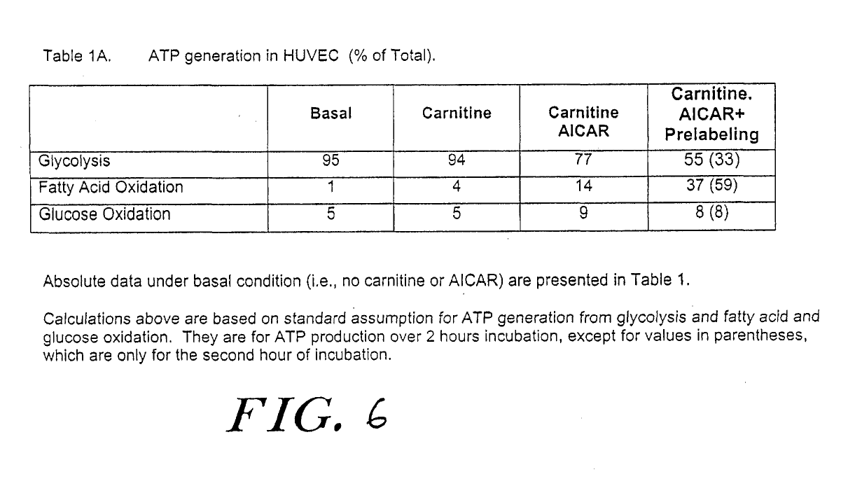

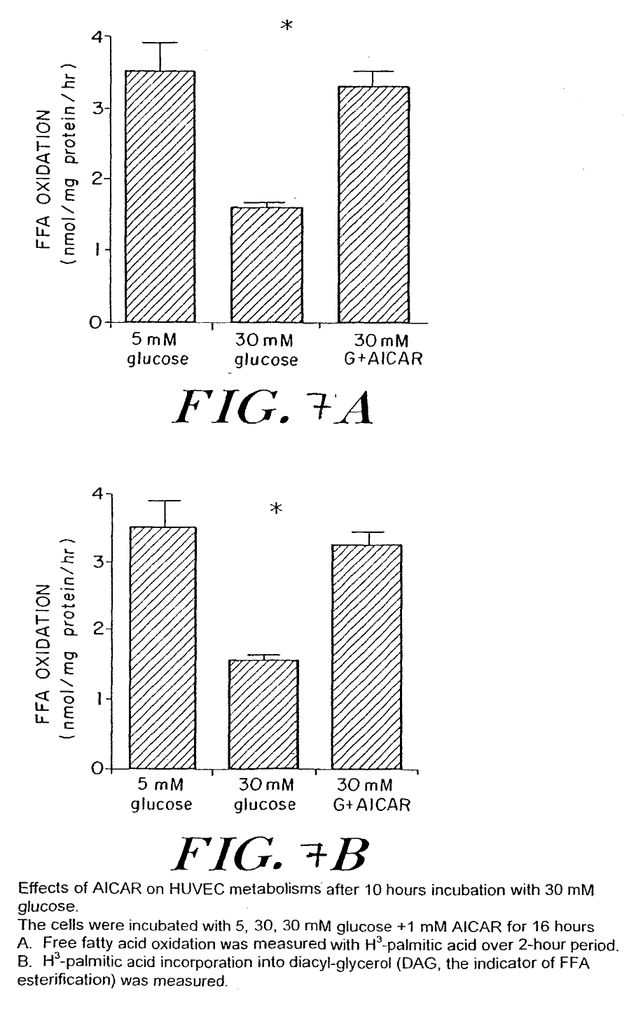

As previously mentioned, AMPK increases fatty acid oxidation in both liver and skeletal muscle by phosphorylation and inhibition of acetyl CoA carboxylase. We wanted to test if this effect of AMPK is also present in endothelial cells. Upon incubation with AICAR, ACC activity decreased. A significant decrease was seen within the first 30 minutes of incubation (FIG. 3). ACC activity went down from 163.1±27.0 pmol/min/mg protein at 0 minutes to 61.1±1.1 at 30 minutes (P=0.004), 73.0 ±12.4 at 60 minutes and 51.2±5.0 at 120 minutes (P=0.015). As previously described, ACC activation increases malonyl CoA levels and this in turn inhibits CPT1, the enzyme responsible for the uptake of long chain fatty acyl CoA into the mitochondria where they are oxidized. If ACC activity goes down, as seen in FIG. 3, one would expect the concentration of malonyl CoA to decrease and fatty acid oxidation to go up. In the absence of carnitine (Table 1, FIG. 4), fatty acid oxidation did not show any changes. However, when 50 pM carnitine was added, fatty acid oxidation increased significantly (FIG. 5). Fatty acid oxidation was measured by the formation of titrated water (³H₂O) in cells incubated with ³H-palmitate. A significant increase was seen only after a 2-hour incubation (P=0.002). In addition, the increase in fatty acid oxidation rose exponentially after 60 minutes of incubation for both + and AICAR. (See Table 1A, FIG. 6.) Glucose and fatty acid oxidation, glucose uptake and lactate and pyruvate release were determined in the absence and presence of 50 pM carnitine. The presence of carnitine had an effect only on fatty acid oxidation. Changes in glucose uptake and oxidation and lactate and pyruvate release caused by AICAR were the same in both the presence (50 pM) and absence of carnitine.

It has been demonstrated, in contracting muscle and muscle incubated with AICAR, that glucose uptake increases as AMPK activity increases, thereby providing more fuel for the muscle cell. To see if this also occurred in endothelial cells, glucose uptake was measured (Table 1). Our results were the opposite of those seen in the muscle, in that glucose uptake was decreased during the AICAR incubation.

In addition, lactate and pyruvate production were measured to see if the rate of glycolysis had changed. Pyruvate production showed a slight, but significant decrease whereas lactate production showed an even more obvious decrease (Table 1).

Despite the decrease in glycolysis (as evident from the decrease in lactate and pyruvate production), the levels of ATP measured by bioluminescence in HUVEC after AICAR incubation, if anything, rose. This was not attributable to an increase in glucose oxidation. Glucose oxidation was measured by determining labeled CO₂ production when the cells are incubated with ¹⁴C-(U) glucose. After two hours, glucose oxidation increased significantly; however, the amount of ATP generated by glucose oxidation was small (See Tables 1 and 1A). The retrieval experiments with labeled bicarbonate showed about 82% retrieval of bicarbonate from the media.

Despite the maintenance or even increase in ATP levels in HUVEC incubated with AICAR, the calculated rate of ATP generation was significantly diminished (See Table 1). Later studies revealed that this was because we underestimated the rate of fatty acid oxidation. Prior studies in skeletal muscle had revealed that radioactive fatty acids added to an incubation or perfusion medium or even to blood, first have to mix with intracellular lipids before they are oxidized. As a result their rate of oxidation can be greatly underestimated unless the tissue is prelabeled with fatty acid. We found that the same phenomenon also occurs in HUVEC (See FIG. 5 and Table 1A). Thus, when the HUVEC were pre-incubated with radioactive palmitate for 24 hours prior to ¹⁴CO₂ collection (to label the intracellular lipid pools), the measured rate of fatty acid oxidation was increased by 5 fold during the second hour of a two-hour incubation with radioactive palmitate added to the medium. (See Table 1A.) During this time period, calculated ATP production in cells exposed to AICAR was similar to that of cells incubated without AICAR; however, fatty acid oxidation accounted for 60% of the ATP generated vs. only 8% in cells that were neither pre-incubated with radioactive palmitate or treated with AICAR. (See Table 1A.)

We also examined the possibility that ATP utilization decreased by AICAR. One of the major ATP users, the Na⁺/K⁺pump, was investigated. The activity of this pump was tested by measuring the uptake of radioactive rubidium in the absence and presence of ouabain, an Na⁺/K⁺pump inhibitor. The difference in uptake, which is referred to as ouabain-sensitive Rb⁺uptake, is the Na⁺/K⁺pump activity. In this experiment, the cells were incubated with 2 mM AICAR for 2 hours. Total rubidium uptake decreased significantly from 12.9 ±1.5 pmol/min/mg protein to 6.0 ±0.3 pmol/min/mg protein at 0 and 2 mM AICAR, respectively (P=0.001, n=6). When the ouabain sensitive component was calculated, no ,difference was seen between the two incubations, (2.11±0.9 and 3.3±0.9 pmol/min/mg protein), thus leading us to believe that no change in Na⁺/K⁺pump activity had occurred. On the other hand, total rubidium uptake was significantly decreased, suggesting that AICAR diminishes the activity of an as yet unidentified K⁺channel. Incubation of the HUVEC with different AICAR concentrations shows that AMPK activity increased significantly. Within the first 30 minutes, AICAR was able to induce AMPK. The activity of AMPK in the endothelial cells is lower than that seen in the liver and skeletal muscle, but the activation due to the presence of AMP is clear, demonstrating the presence of AMP-activated protein kinase. The AMPK activity remained high with a longer incubation time up to 120 minutes. The 2 mM AICAR was the concentration of choice for the remaining experiments due to its obvious activation of AMPK and its low toxicity to the cells.

One of the roles of AMPK is the sensing of AMP vs. ATP levels in the cell. Activation of AMPK in response to an increase in the AMP/ATP ratio allows the cell to modify its metabolic activities to provide energy when needed. As described above, AMPK phosphorylates acetyl CoA carboxylase, inhibiting its activity, thus reducing the levels of malonyl CoA. ACC is the first committed step in fatty acid synthesis and thus energy storage. In addition, the decrease in malonyl CoA levels relieves the inhibition of CPTI, thus allowing long chain fatty acids to be taken up into the mitochondria where they are oxidized. Thus, modulating the activity of ACC through AMPK not only decreases energy storage but also allows for energy production.

The results described in other tissues are seen in the endothelial cells. FIG. 3 demonstrates both the presence of ACC in the endothelial cells as well as its inhibition upon incubation with AICAR, i.e. activation of AMPK. The presence of ACC is shown by the substantial increase in enzyme activity when 10 mM citrate is present vs. 0 mM citrate. Citrate allosterically activates ACC, but not other enzymes, such as propionyl CoA carboxylase and pyruvate carboxylase, which could also use HCO₃⁻as substrate. Thus, the increase in radioactive HCO₃⁻use in the presence of citrate is a reflection of ACC activity only. Similar to AMPK, the activity of ACC is lower in endothelial cells than in skeletal muscle and liver. Again, within 30 minutes of incubation in 2 mM AICAR, ACC activity is decreased and it remains low for 2 hours.

### EXAMPLE 2

### Effect of AICAR on Apoptosis in Human and Bovine Pericytes

Human and bovine retinal pericytes were plated in 6 well plates, grown in a 37° C., 5% Co₂ incubator with SmBM medium and treated with AICAR. Apoptosis was induced by incubating the cells with a medium containing 0-0.5 mM palmitic acid for 3 days. Apoptotic cells were determined by conventional TUNEL staining. Ceramide and DAG levels were measured the by diacylgycerol kinase method using 32P-ATP. To determine the effects of AICAR, 1 mM AICAR was added for these 3 day periods.

Increased fatty acid levels were added to the medium to promote apoptosis in human and bovine retinal pericytes in a dose dependent fashion (0.5% with 0.1 mM palmitate vs 27% with 0.5 mM palmitate). The apoptotic rate was further increased by incubating the cells with high glucose. (With 0.2 mM palmitate, 4% of the cells were apoptotic in 5 mM glucose vs. 7% of the cells in 20 mM glucose.) This increase in apoptosis was accompanied by increased intracellular ceramide levels (3 nmol/mg protein with 0.1 mM palmiate vs 10 nmol/mg protein with 0.5 mM palmitate). Incubation with 1 mM AICAR decreased apoptosis by 50% (27±4% with 0.5 mM palmitate vs 10±2% with 0.5 mM palmitate+1 mM AICAR). AICAR also decreased ceramide levels by 60% (9±2 nmol/mg protein with 0.5 mM palmitate vs 3.8±1 nmol/mg protein with 0.5 mM palmitate +1 mM AICAR) in human retinal pericytes and DAG levels by 50% (47±3 nmol/mg protein with 0.5 mM palmitate vs. 24±5 nmol/mg protein with 0.5 mM palmitate +1 mM AICAR).

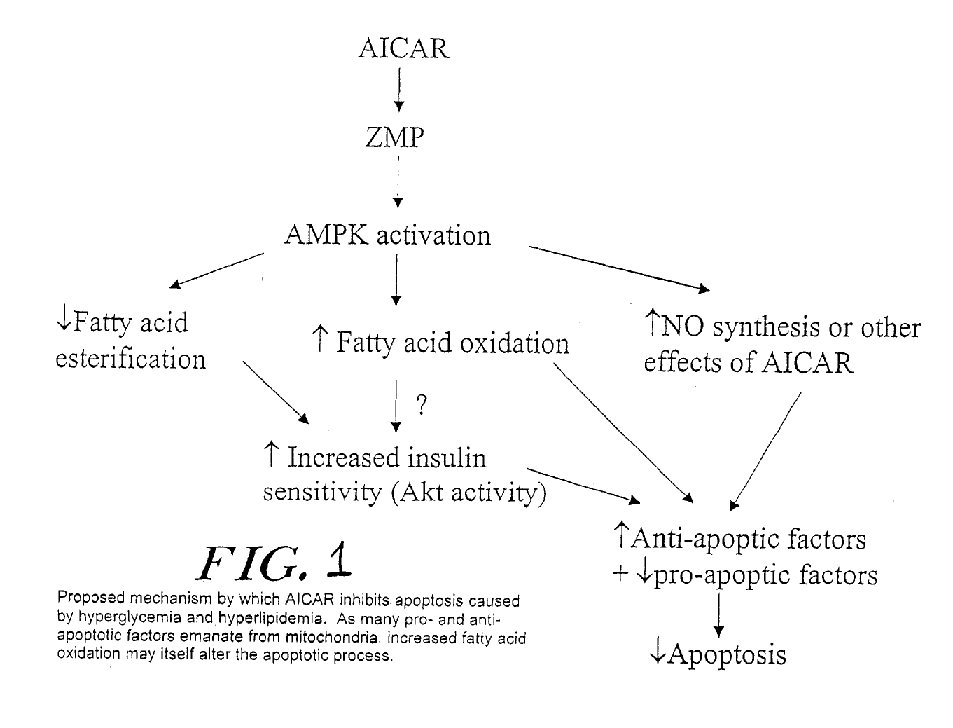

These results are consistent with the metabolic effects of AICAR depicted in FIG. 1. They show that increased glucose levels enhance fatty-acid induced apoptosis, possibly by enhancing free fatty acid esterification (DAG and ceramide), and that AICAR prevents these effects.

### EXAMPLE 3

### Effects of AICAR on Mitochondrial Membrane Potential, Free Fatty Acid Oxidation and Free Fatty Acid Incorporation Into Diacylglycerol in HUVEC—Relationship to Apoptosis Levels

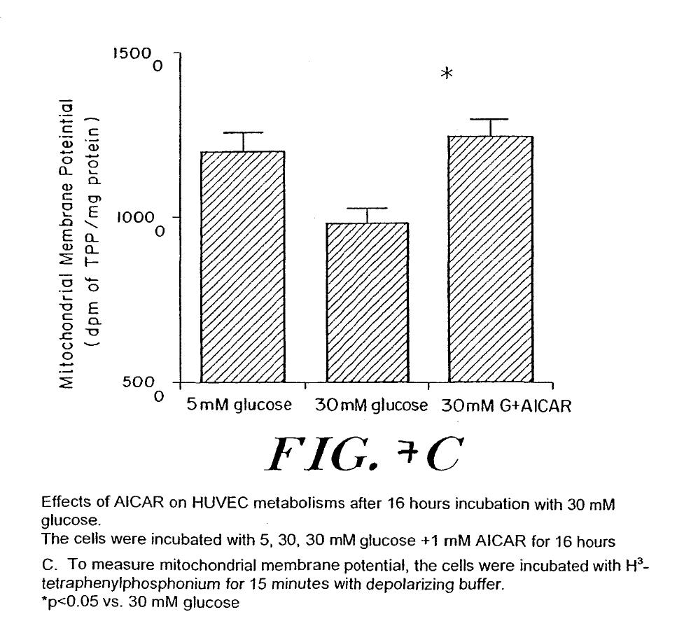

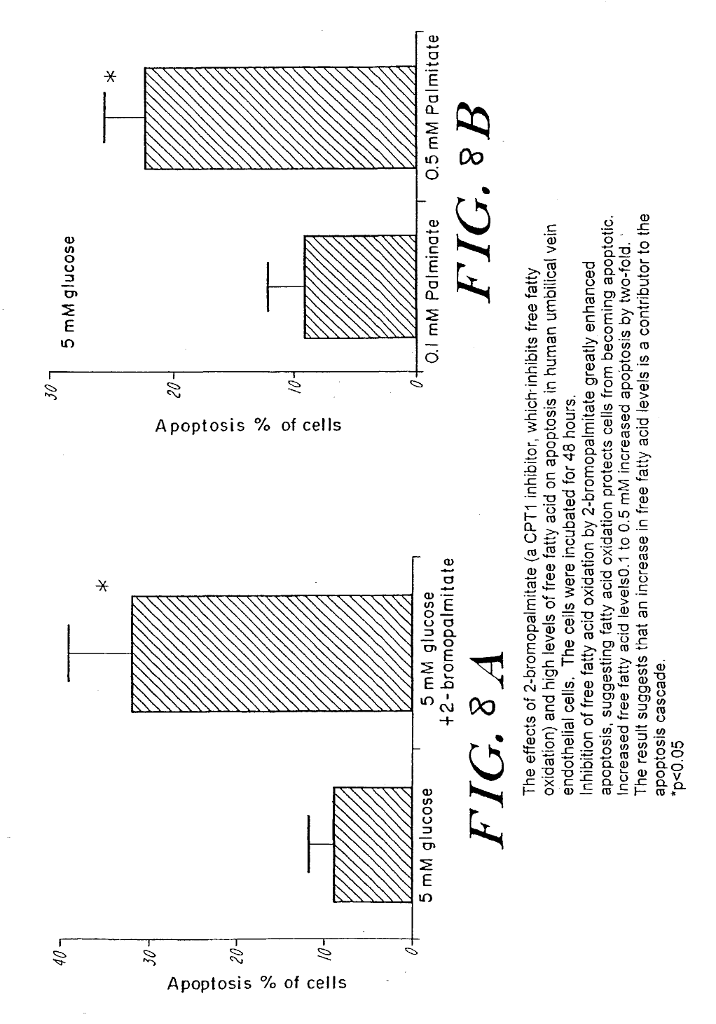

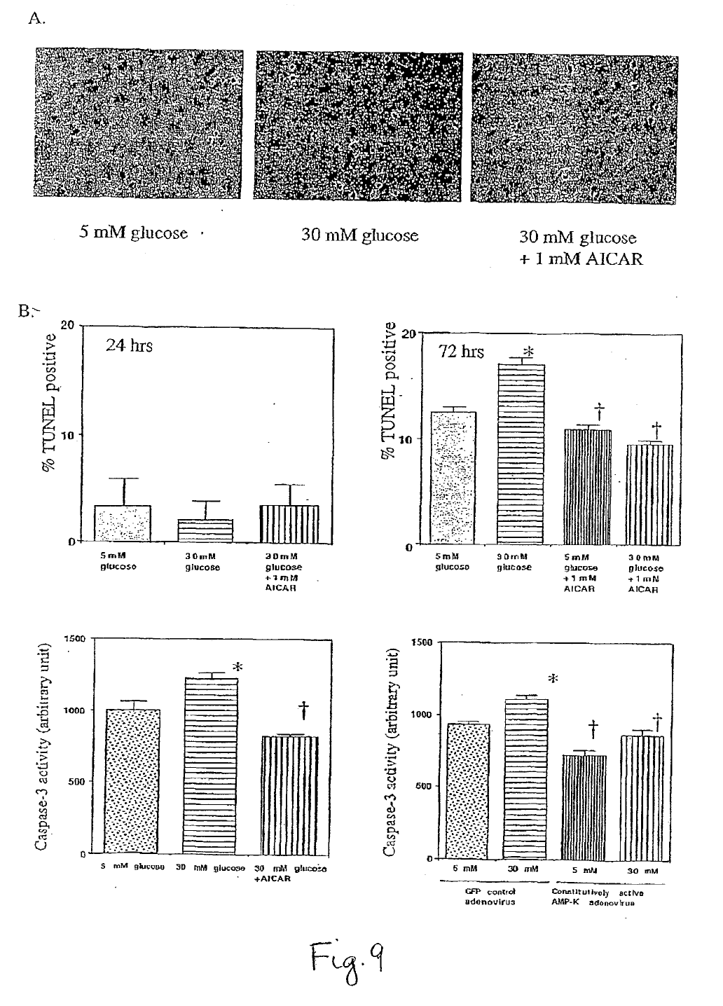

Incubation of HUVEC with 30 mM glucose for 16 hrs decreased free fatty oxidation by 50% (FIG. 7A) and increased free fatty acid incorporation into diacylglycerol (DAG) (i.e., esterification) by 40% (FIG. 7B). These changes were accompanied by a significant decline in mitochondria membrane potential (FIG. 7C). Treatment with 1 mM AICAR in 30 mM glucose prevented all these changes.

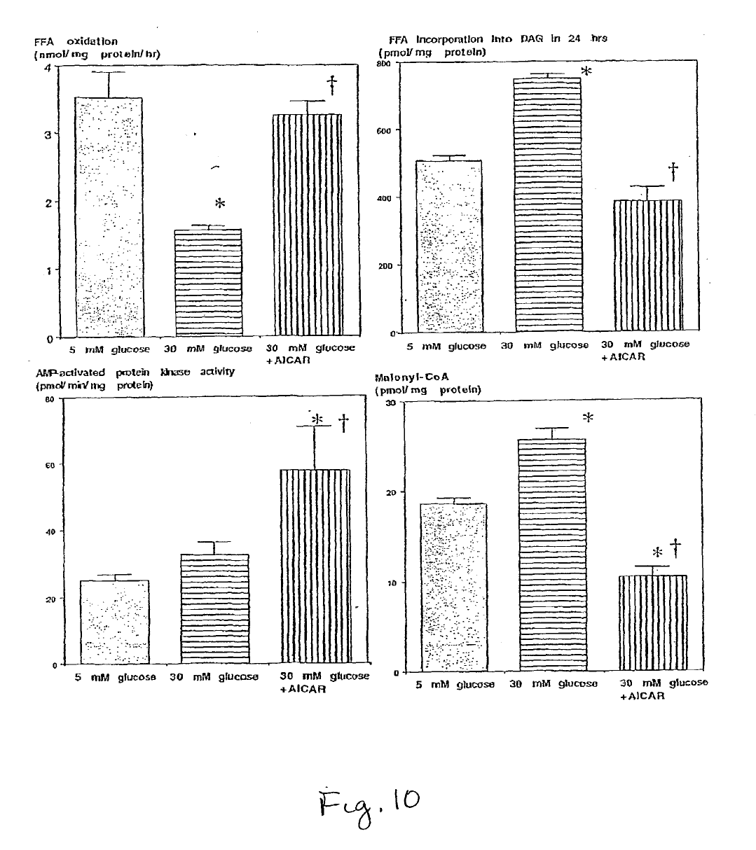

The effects of AICAR on HUVEC in 30 mM glucose were consistent with the scheme depicted in FIG. 1, in which a high glucose concentration increases apoptosis by decreasing free fatty-acid oxidation and increasing esterification. AICAR prevented these changes as well as the decreased mitochondrial membrane potential caused by hyperglycemia. Because decreased mitochondrial membrane potential promotes cell apoptosis, this observation further suggests that a decrease in fatty-acid oxidation and an increase in the esterification of fatty-acids cause apoptosis. To test this notion, experiments were performed in HUVEC incubated with 2-bromopalmitate, an inhibitor of CPT1 which inhibits free fatty oxidation, or a higher concentration of free fatty acids (0.5 mM vs. 0.1 mM palmitate). As shown in FIG. 8A, inhibition of free fatty acid oxidation by 2-bromopalmitate promotes apoptosis, as does incubation with higher levels of palmitic acid (FIG. 8B). These results are consistent with the proposed metabolic theory of apoptosis and the effects of AICAR to prevent it.

### EXAMPLE 4

### Hyperglycemia-Induced Apoptosis is Inhibited by Aicar and by Expression of Constitutively Active AMP-Kinase.

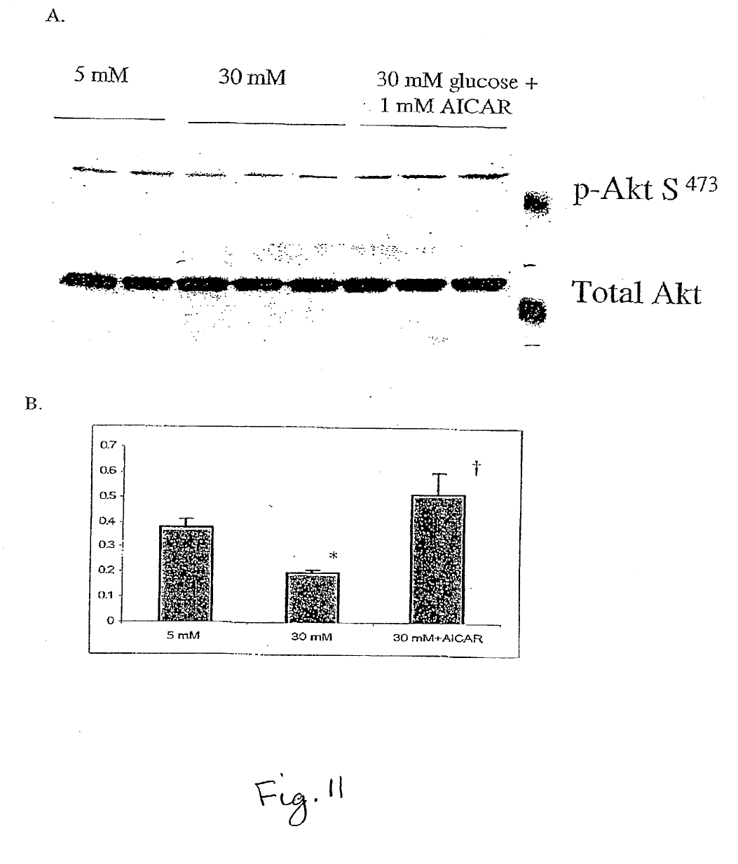

As shown in FIG. 9, the percentage of TUNEL-positive cells was similar (3-4%) in HUVEC incubated for 24 hours in the EBM-2 media containing 5 or 30 mM glucose. In addition, no differences in cell number, morphology or protein were observed at this time. By 72 hours, however, a significantly greater percentage of cells (18 vs 13% p<0.05) incubated at the higher glucose concentration were apoptotic. Also shown in FIG. 9, AICAR had no affect on apoptotic rate in cells incubated in 30 mM glucose after 24 hours, but it completely prevented the increase in apoptosis caused by hyperglycemia at 72 hours. No increase in apoptosis was observed at 72 hours in an osmotic control in which cells were incubated with 5 mM glucose and 25 mM mannitol, in agreement with the findings of others.

As shown in FIG. 9C, an increase in caspase-3 activity, which is thought to be an early signal of apoptosis, was evident in cells incubated with 30 mM glucose for 24 hr (1225±42 arbitrary unit vs 1002±61 for 5 mM glucose, p<0.01, n=5), and it, too, was completely prevented by addition of AICAR (833±14 arbitrary unit, p<0.01 vs 30 mM glucose). A similar effect was observed when AMPK activity was increased by infecting HUVEC with constitutively active AMPK using an adenoviral vector (FIG. 9C).

### EXAMPLE 5

### Sustained Hyperglycemia Alters Intracellular Fatty-Acid and Glucose Metabolism and Akt Activation, But Does Not Alter Ceramide Content or de novo Synthesis

We have previously shown that incubation of HUVEC for 2 hr in media containing 30 mM vs 5 mM glucose causes no changes in AMPK activity, malonyl CoA concentration, fatty acid oxidation, or lactate release. In contrast, incubation at this glucose concentration for 24 hr resulted in increases in the concentration of malonyl CoA, diminished fatty acid oxidation and an increased incorporation of both radioactive glucose and radioactive palmitate into diacylglycerol (FIG. 10). In addition, it impaired Akt phosphorylation in the presence of insulin (FIG. 11). Like the decrease in mitochondrial membrane potential and increases in caspase-3 activity caused by hyperglycemia, these changes were completely prevented by incubation with AICAR; indeed, the concentration of malonyl-CoA was even lower than that in cells incubated with 5 mM glucose. AMPK activity tended to be higher after 24 h of incubation with 30 mM glucose; however, whether it was inappropriately low, in light of the decrease in ATP content (and presumably an increased AMP/ATP ratio), was not ascertained.

To assess whether alterations in ceramide contributed to these events, and particularly the decrease in membrane potential, we attempted to measure ceramide content. Ceramide was not detectable in HUVEC incubated with either 5 or 30 mM glucose. In contrast, HUVEC incubated in a medium containing 5 mM glucose and 0.1 mM palmitate for 24 hr showed a ceramide band on autoradiography, but no increase in TUNEL staining. Consistent with these findings, we found no increase in serine incorporation into ceramide, (presumably a measure of de novo synthesis) in cells incubated for 24 hr in 30 vs 5 mM glucose. Observed values were (8.4±0., 10.8±0.4, and 9.0±1.2, (n=4), in cells incubated in 5, 30 mM glucose and 30 mM glucose+AICAR, respectively).

### EXAMPLE 6

### AICAR Mimics the Positive Effects of Exercise In Preventing or Treating Obesity With Minimal If Any Negative Side Effects

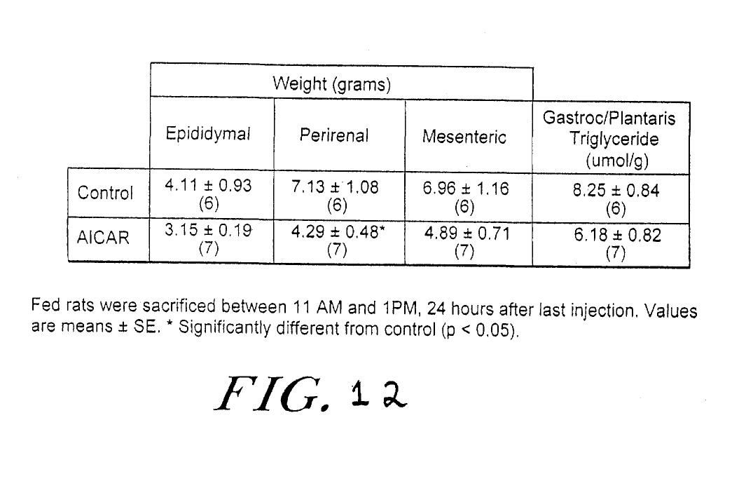

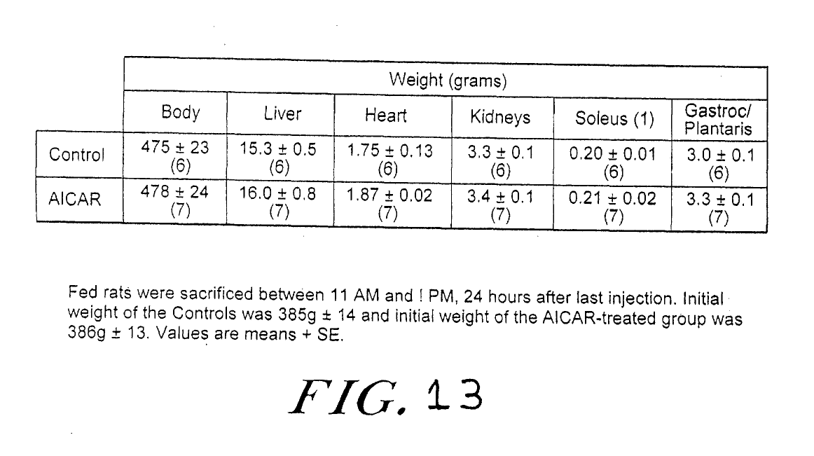

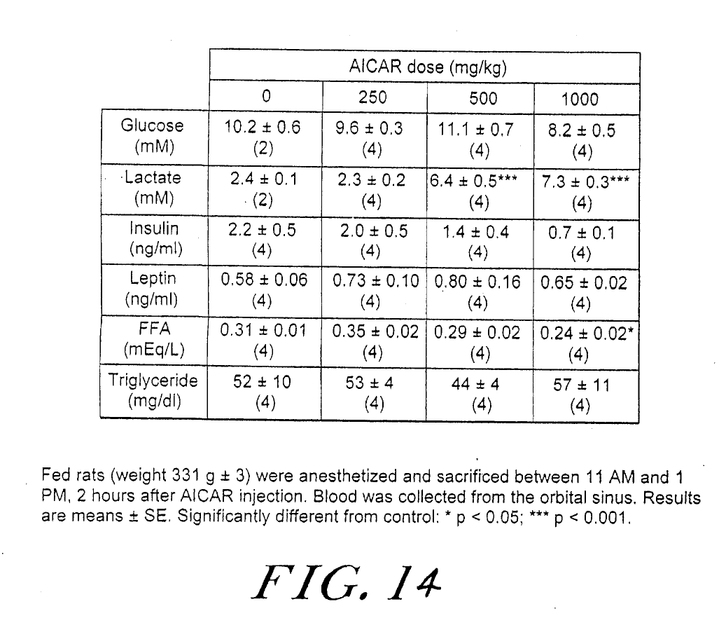

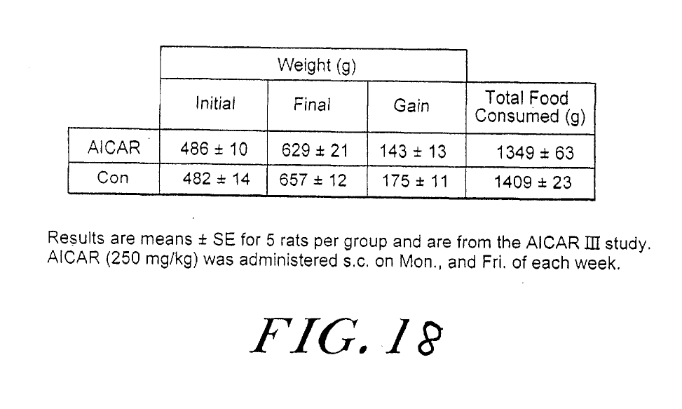

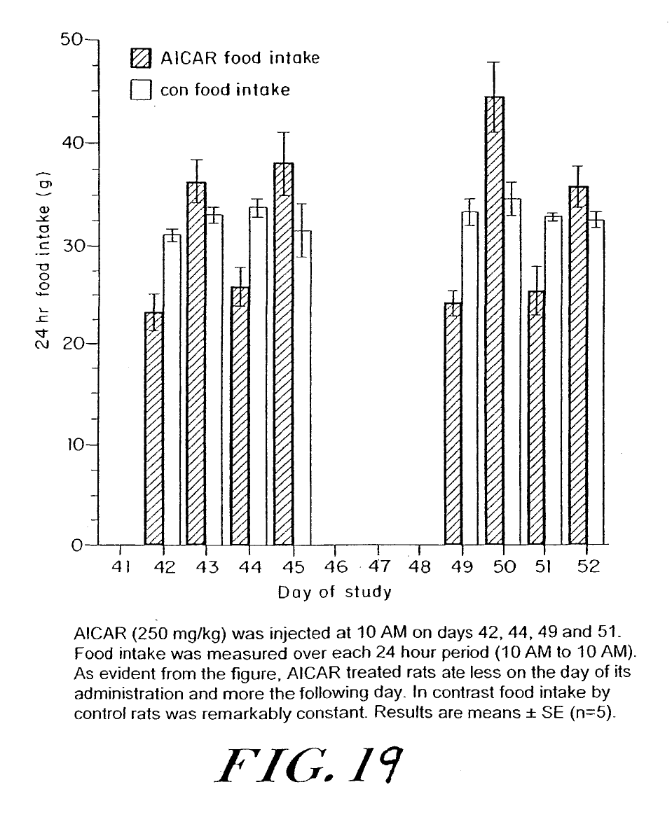

In experiments with juvenile rats, each weighing about 400 gms, AICAR was administered subcutaneously at a dose of 250 mg/kg on M, W, F of each week for 25 days. This dose distributed in the extracellular space of the rat (approx. 20 ml/100g b. w.) yields a concentration in the extracellular space of approximately 5 mM. At postmortem evaluation, e.g., as shown in Table 2, FIG. 12, the mass of specific intra-abdominal (retroperitoneal, mesenteric, epididymal) fat depots was diminished by 20-40% and muscle triglycerides by approximately 20%. As shown in Table 3, FIG. 13, no differences were observed in the weights of the heart, liver or other organs in AICAR treated rats at 25 days; however, an increase in muscle mass is suggested at 106 days (Table 6, FIG. 16).

Referring to FIG. 19, it can be seen that food intake was diminished by approximately 25% over the first 24 hrs following each injection. The rats appear to compensate by eating more on the following day. This pattern only became evident after 1-2 weeks of AICAR administration, although it and continued indefinitely thereafter. Thus, the anorexigenic action of AICAR does not appear to be due to an acute aversion to food. Cumulative net food intake was diminished only modestly (about 3%) during all long-term studies.

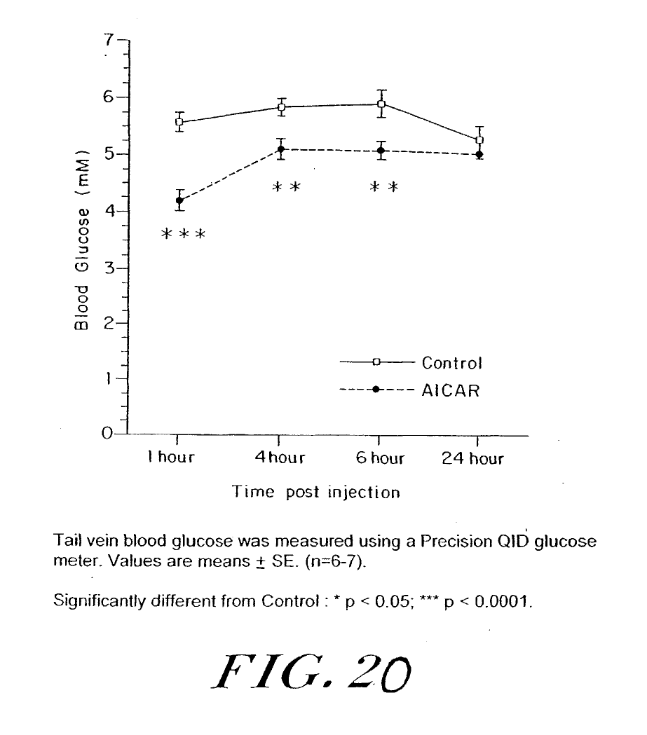

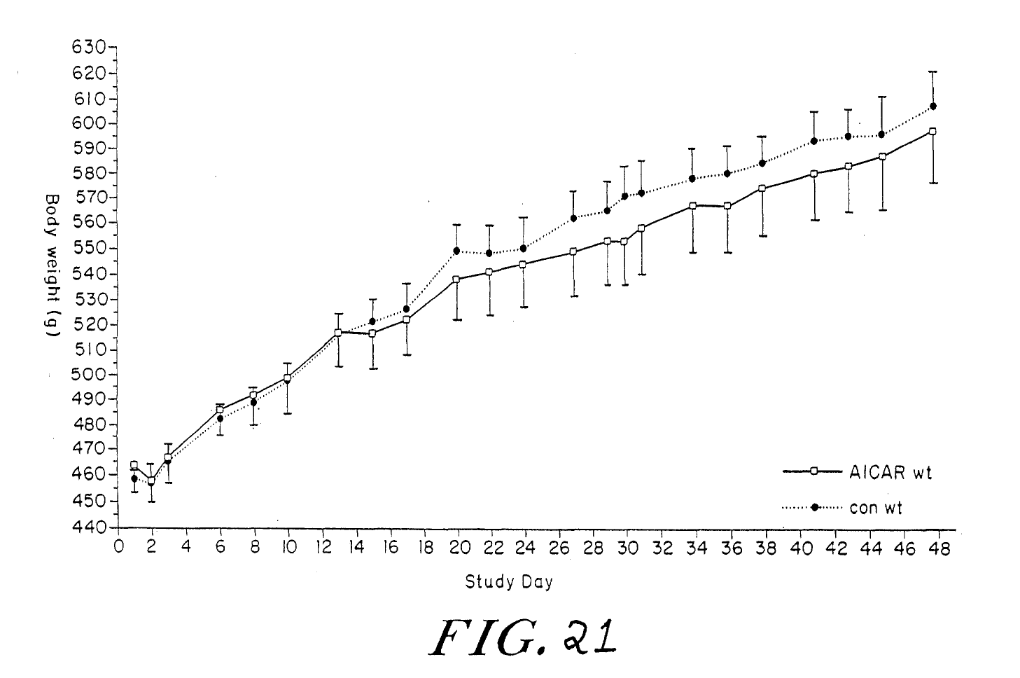

AICAR moderately diminished blood glucose levels 2-6 hours following its administration (FIG. 20). At high doses, the acute effect of AICAR was to increase blood lactate and decrease plasma free fatty acid (FFA) levels (Table 4, FIG. 14). AICAR at all doses studied did not significantly alter plasma triglycerides or leptin at 2 hrs (Table 4).

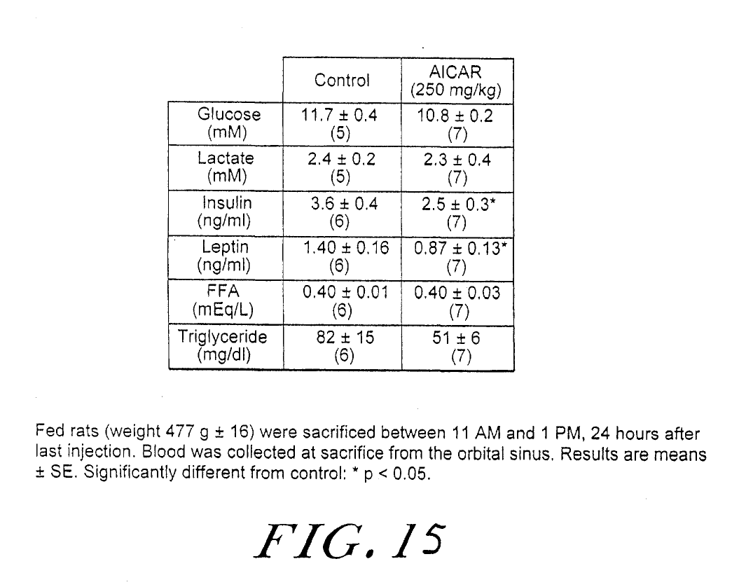

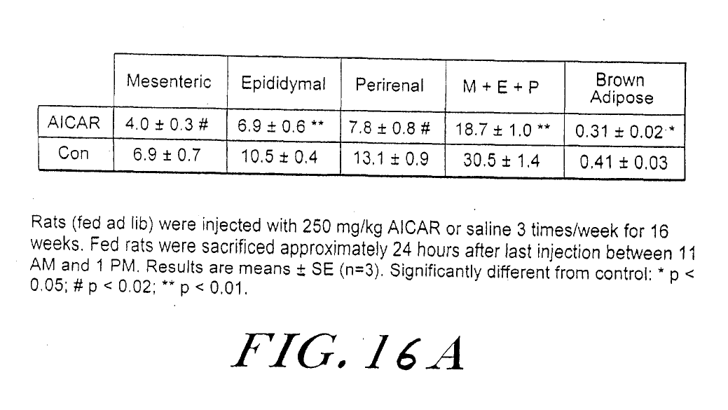

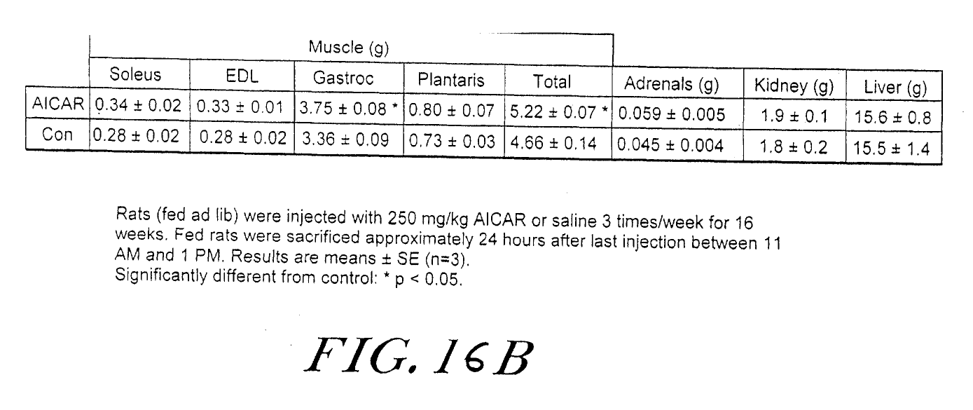

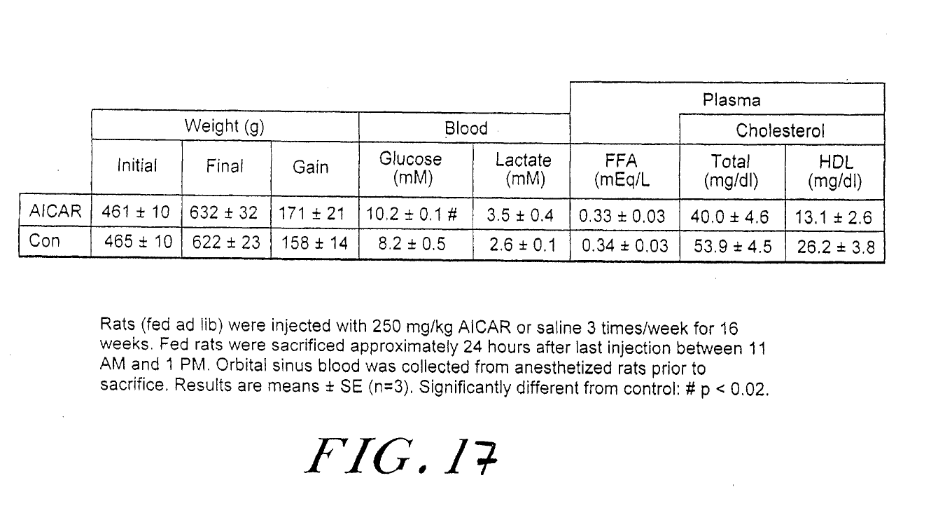

Referring to Table 5, FIG. 15, in rats chronically treated with AICAR (e.g., for 25 days), plasma leptin was significantly diminished at 24 hrs following the last injection. This is in keeping with the diminished adiposity of the treated subjects. Muscle triglyceride was reduced by 25% (Table 2) and plasma triglycerides by 40% (Table 5).

Table 8, FIG. 18, shows that food intake was diminished by 3-4% during a 12-week study period and that weight gain was less by 32 g in the AICAR group. Due to interanimal variability and the small number studied, however, neither difference was statistically significant.

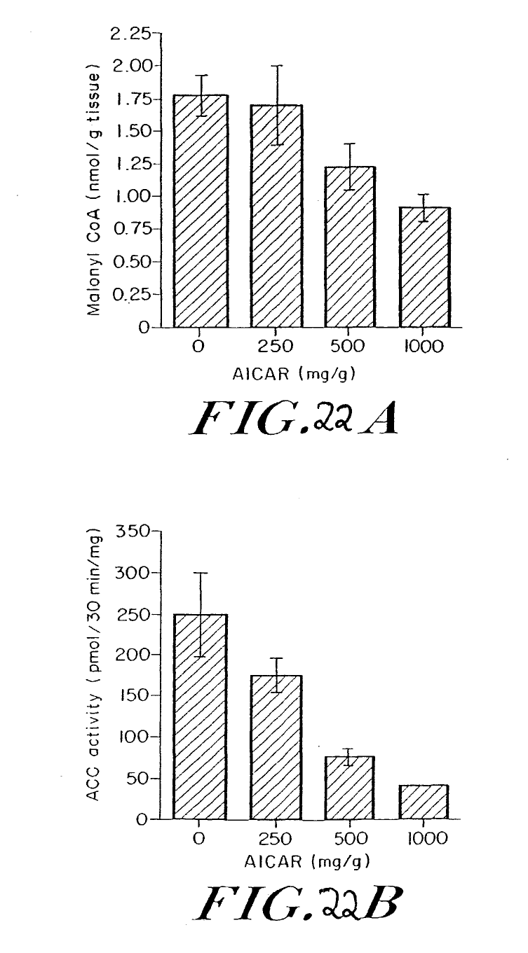

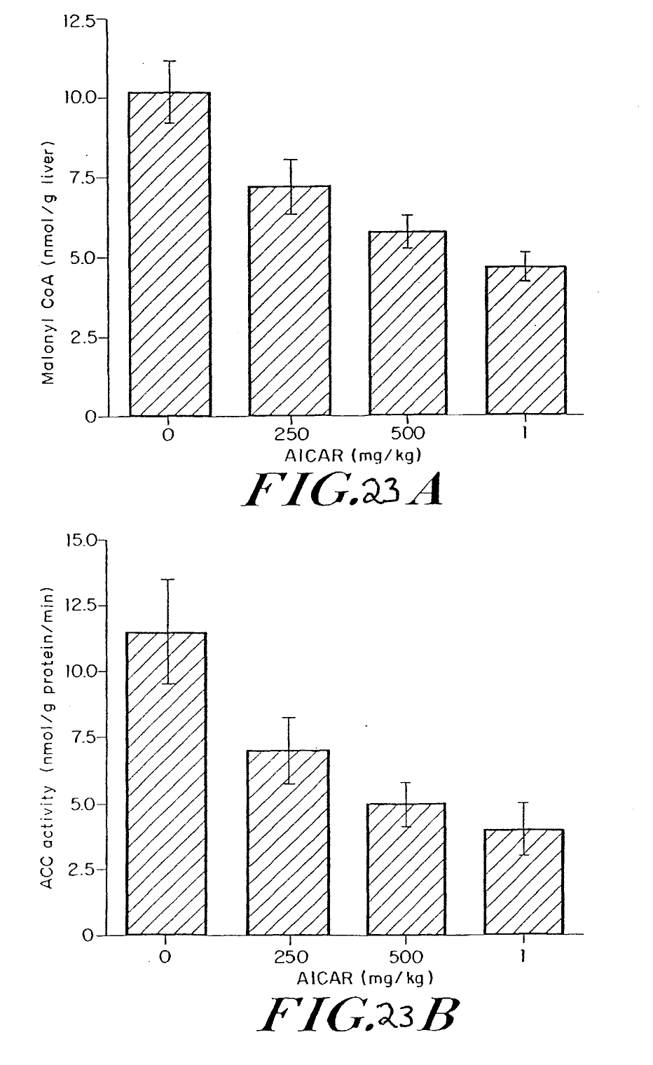

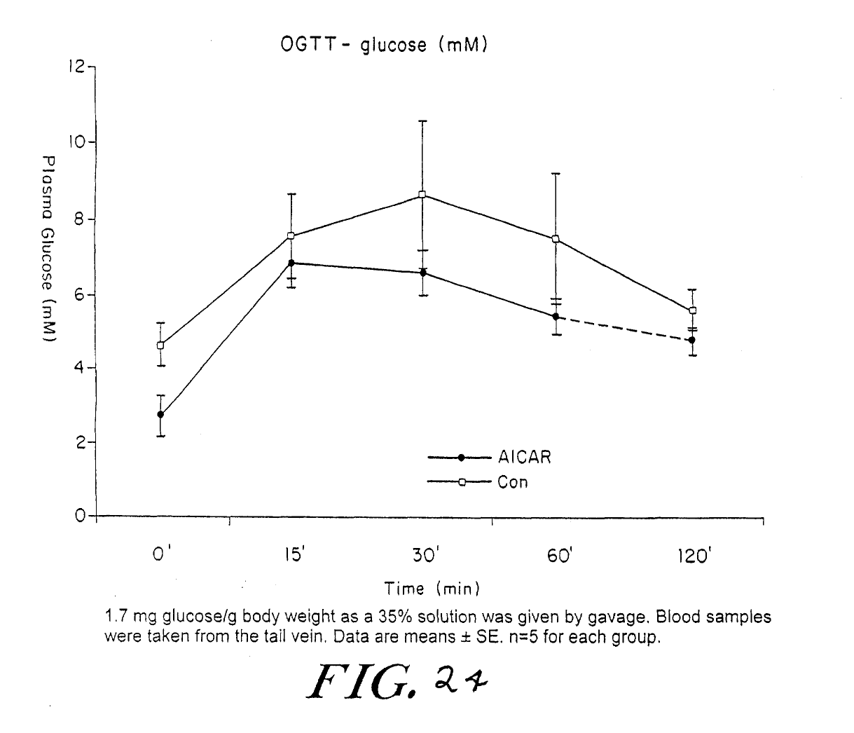

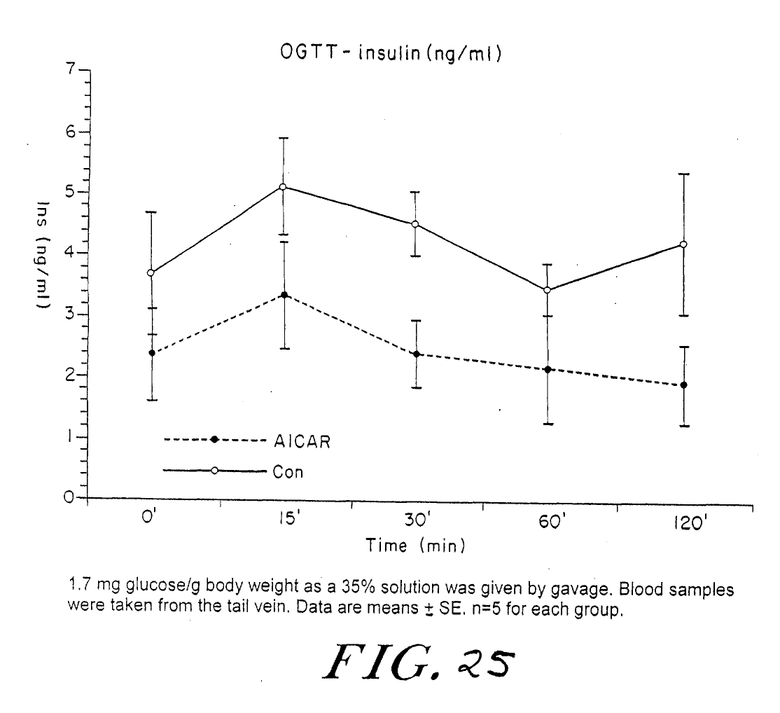

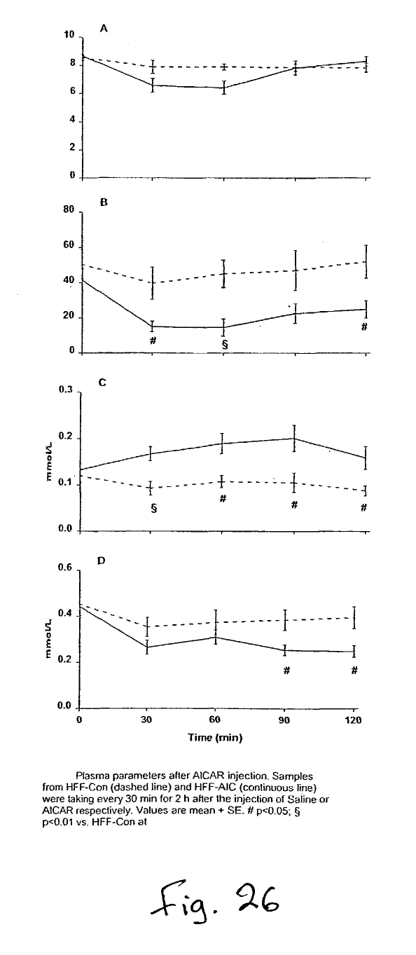

Measurement of the activity of ACC and malonyl CoA concentration in liver and muscle two hours after AICAR injection revealed the site of AICAR action. Identical changes to those in liver were seen in fat cells. Dose response curves are shown in FIGS. 22 and 23. FIG. 23 shows that AICAR significantly diminished both ACC activity and malonyl CoA concentration in liver when administered at a dose of 250 mg/kg, although a maximal effect was not observed until a higher dose (500 mg/kg) was administered. In contrast, at the 250 mg/kg dose, AICAR had no effect on malonyl CoA levels in muscle and it did not depress ACC significantly, although a trend for ACC to be diminished was evident.

Winder et al. (J. Appl. Physiol. 88:2219-2226, 2000) have shown that AICAR administration at a daily dose of 1000 mg/kg/day for 28 days induces the protein expression or activity of a number of molecules in skeletal muscle. These include the GLUT 4 glucose transporter, hexokinase and various mitochondrial enzymes including cytochrome C oxidase and citrate synthase. These investigators also found that AICAR therapy at the dosage used increased muscle glycogen. In contrast, it was observed here, in experiments supporting the method of the invention, that when RT-PCR (GLUT 4, UCP3 and cytochrome C oxidase) and direct enzyme activity measurement (citrate synthase) were used as indicators, no changes were found in these parameters using a 250 mg/kg AICAR dose administered intermittently, e.g., 3 days/wk over 106 days. Also we found no increase in muscle glycogen.

Winder (ibid.) noted hepatic enlargement in rats treated with AICAR at a dose rate of 1000 mg/kg body weight daily for 28 days. Although Winder also noted some decrease in food intake in these rats, it was not possible to determine whether this was due to hepatotoxicity. In addition, Winder pair-fed the control rats so that their food intake matched that of the AICAR-treated rats. In contrast, no hepatic enlargement was found with the AICAR dosage regimen used here according to the method of the invention. Also, no evidence of hepatocellular damage was observed morphologically (light microscope).

Increases in abdominal fat, particularly intra-abdominal fat, are associated with insulin resistance and increases in muscle triglyceride. Therefore, the effect of chronic AICAR treatment, at the dosage used, on plasma insulin levels and glucose tolerance were assessed. As shown in Table 4, plasma insulin levels were significantly diminished in rats treated with AICAR. In addition, lower glucose and insulin levels were observed during an oral glucose tolerance test (FIGS. 24 & 25).

### EXAMPLE 7

### Acute in vivo Administration Of Aicar to Fat-Fed Rats Diminishes Insulin Resistance in Muscle and Liver

Acute Studies

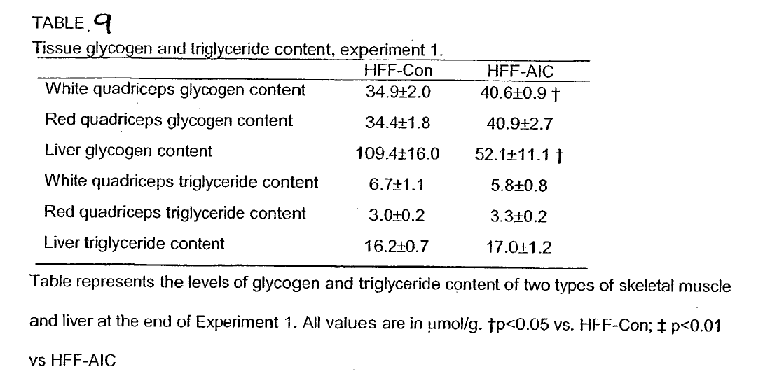

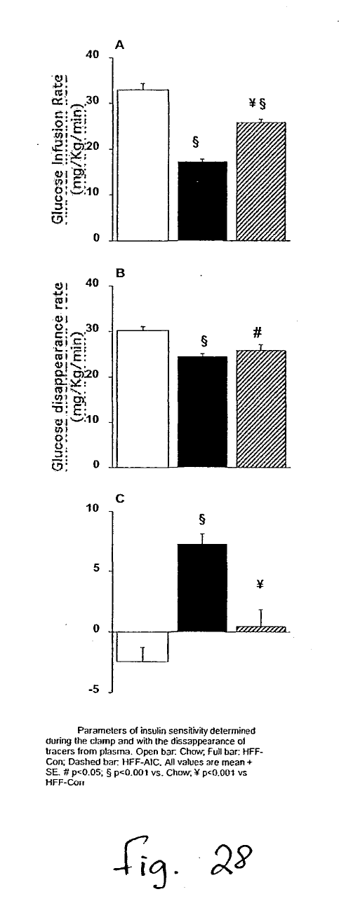

In this study, we investigated the effects of a single injection of AICAR 24 h later on the insulin sensitivity of rats made insulin resistant by ingestion of high-fat diet (HFF) for 3-4 weeks. In doing so, we clearly demonstrated the in vivo influence of the pharmacological activation of AMPK in insulin sensitivity. (See clamp studies.) As a preliminary step in these investigations, we carried out experiments in which the acute effects of AICAR on these rats at 30-120 min post-injection were examined. Plasma parameters: This experiment was performed on HFF rats under conscious conditions following 5-7 hours of fasting. After catheters were connected to blood sampling syringes, the animals were allowed to rest for 40-50 min. Two blood samples were taken 20 min. apart, constituting the basal samples (time 0). The animals then received a subcutaneous injection of either AICAR (250 mg/kg body weigh in saline) (HFF-AIC) or an equivalent amount of normal saline (HFF-Con) and were returned to their cages. Blood samples were collected at 30, 60, 90 and 120 min. after the injection. After the last sample was taken, the animals were euthanized. There were no differences in the body weight (HFF-AIC: 368+4; HFF-Con: 364+7 g), food intake or plasma parameters between the groups before the injection (FIG. 26, Time 0). As shown in FIG. 26A, the injection of AICAR (HFF-AIC) caused within 30 minutes a decrease in plasma glucose that lasted at least 60 minutes. This was accompanied by a decrease in plasma insulin to values significantly lower than in the saline injected (HFF-Con) control group (FIG. 26B). While the levels of plasma FFA remained unchanged throughout the study in both groups, the treatment caused an increase in plasma glycerol and decrease in plasma triglyceride levels (FIGS. 26C and D, respectively) compared to HFF-Con rats. Tissue parameters: The glycogen and triglyceride content of two types of skeletal muscle and liver at the end of the experiment were determined (Table 9, FIG. 27). HFFAIC rats showed a 16% increase in glycogen content in white muscle 2 h after the injection. Similar values were obtained in red skeletal muscle (p=0.08 vs. HFF-Con). In contrast, liver glycogen was significantly reduced, by 52%, after the AICAR injection. Changes in tissue triglyceride content in HFF-AIC rats were not significant.

Clamp Studies

HFF rats were randomly assigned to receive a subcutaneous injection of either AICAR (250 mg/kg in saline) (HFF-AIC) or an equivalent volume of normal saline *(HFF-Con) in the same way as described above. They were then returned to their cages and given a high-fat meal. The injection of AICAR caused a minor reduction in the food eaten overnight but without any impact on body weight. To avoid the possible effects of a reduced caloric intake, the HFF-Con animals were pair-fed with HFF-AIC rats. 24 h after the injection, and after a 5-7 h fasting period, the rats were submitted to an euglycemic hyperinsulinemic clamp, to determine the whole body insulin sensitivity.

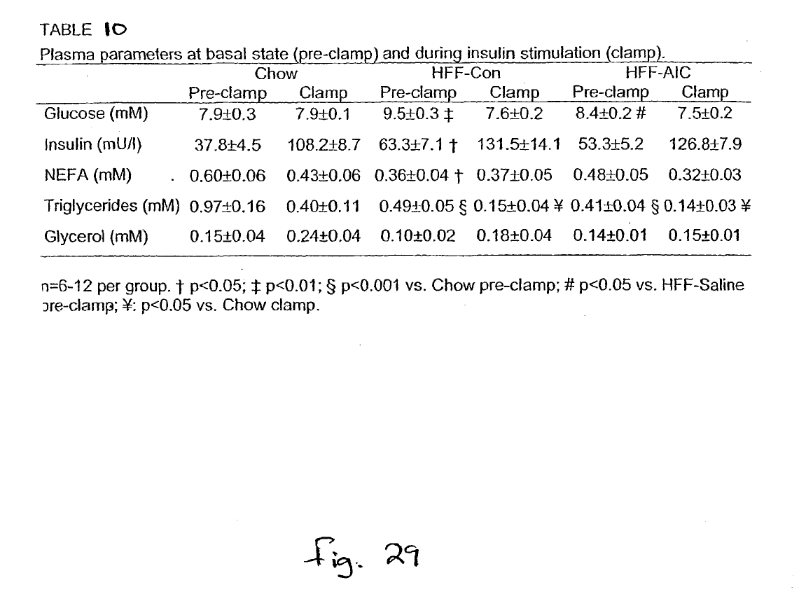

The parameters of whole body insulin sensitivity are shown in FIG. 28. The high-fat diet caused a decrease in the glucose infusion rate (GIR) required to maintain euglycemia during hyperinsulinemia. Prior AICAR administration increased GIR by 50%. Rd was reduced in both groups of high-fat fed rats (FIG. 28); HFF-AIC rats showed a slight but not significant increase in this parameter. Insulin completely suppresses the HGO in the Chow rats (FIG. 28C). In HFF-Con rats, insulin was unable to suppress HGO. However, pretreatment with AICAR significantly ameliorated this effect, reducing HGO to close to the normal value (p>0.05 vs. Chow).

Table 10, FIG. 29, summarizes plasma metabolites in the basal state and during clamp. In basal conditions, the high fat diet caused an increase in plasma glucose and insulin, and a decrease in plasma FEFA and triglycerides. The prior administration of AICAR decreased the hyperglycemia caused by high-fat feeding, but did not alter the other parameters when compared to HFF-Con rats.

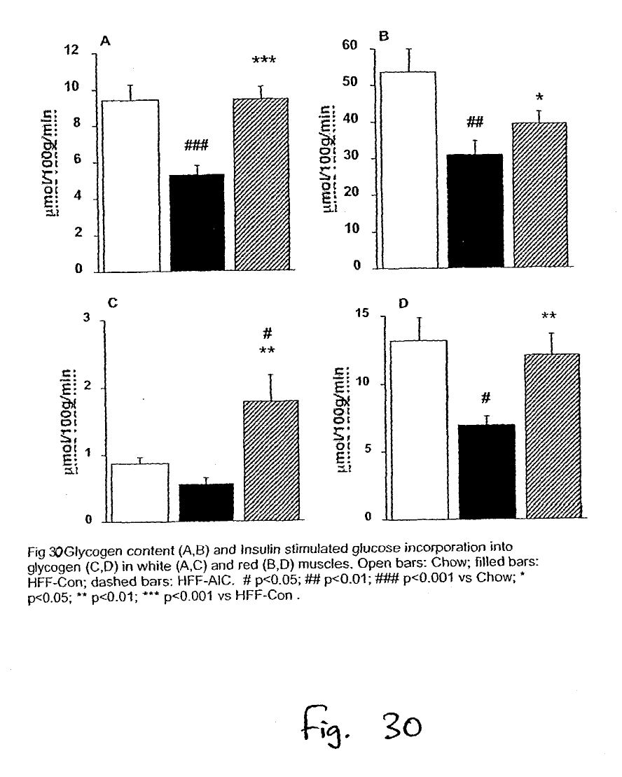

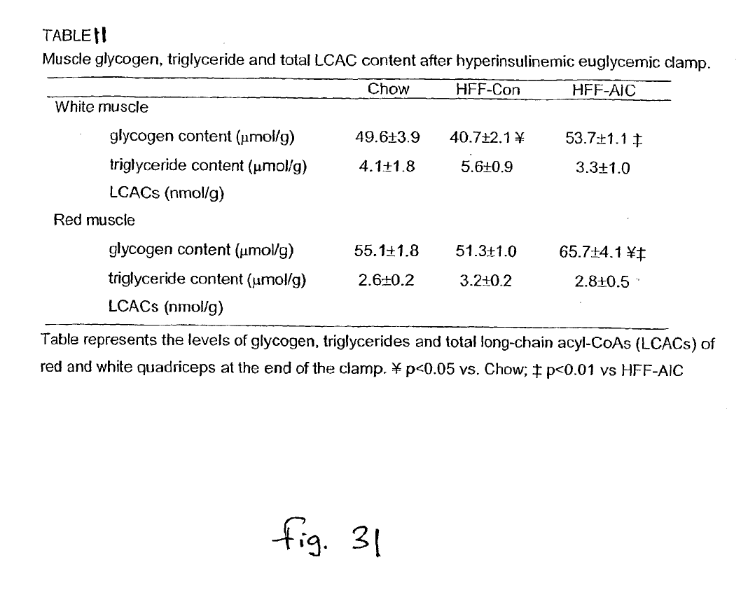

The effects of AICAR on muscle are shown in FIG. 30. AICAR enhanced Rg' (rate of glucose uptake) in white muscle by 79% in HFF-rats, to values similar to those in Chow fed rats (FIG. 30A). There was also a tendency of increased Rg' in red muscle (p=0.085, FIG. 30B).

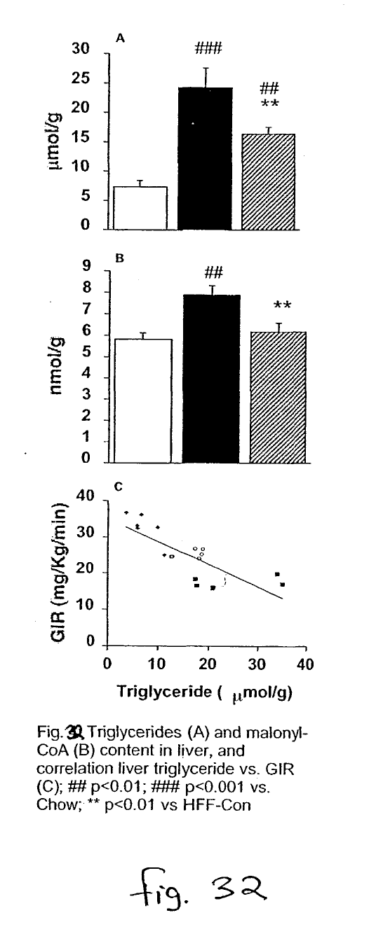

In HFF-AIC rats, insulin-stimulated glucose incorporation into glycogen was also significantly increased in both white and red muscle following AICAR treatment(FIG. 30C,D). A similar trend was found in insulin-stimulated glucose incorporation into lipids. These results were consistent with the finding of increased glycogen content at the end of the clamp (Table 3). There was no significant change in the levels of muscle triglycerides or long chain fatty acyl CoA (LCFACoA) (Table 11, FIG. 31).

After the clamp, malonyl-CoA in red muscle was reduced by 44% in the HFF-AIC group, to a value similar to that of Chow rats (Chow: 1.13±0.09; HFF-Con: 1.83±0.13 p<0.01 vs. Chow; HFF-AIC: 1.03±0.12 nmol/g, p<0.001 vs. HFF-Con).

FIG. 32 shows triglyceride and malonyl-CoA content in liver after clamp. Compared to the Chow group, liver triglycerides were elevated in HFF-Con and this accumulation was reduced by 33% by AICAR pretreatment (FIG. 32A). There was also a 21% decrease in malonyl-CoA in HFF-AIC group compared to the HFF-Con control (FIG. 32B). The levels of liver triglycerides was inversely correlated with GIR (r=−0.78, p<0.001; FIG. 32C). The rates of glucose incorporation into glycogen and into triglycerides in liver were reduced in both HFF groups when compared to Chow.

This study demonstrates for the first time the sustained insulin sensitizing effects of AICAR 24 h after administration in rats fed a high-fat diet. The prolonged enhancement of insulin sensitivity was found in both muscle and liver. These insulin sensitizing effects were achieved at a dose much lower than doses used in in vivo studies by others. We have thus observed that, in insulin-resistant, high-fat-fed (HFF) rats, AICAR administered 24 h previously still enhances the ability of insulin to suppress hepatic glucose production and to increase muscle glucose transport and glycogen synthesis. These results provide theoretical support for the benefit of intermittent administration of this therapeutic agent.

From these results, we can also conclude that a single dose of the AMPK activator AICAR leads to an improvement in whole body, muscle and liver insulin sensitivity in high-fat fed rats well beyond the expected time range of activation of AMPK. Also enhanced insulin-mediated glycogen synthesis caused by AICAR occurs in both red and white muscle and does not depend on a period of prior glycogen depletion. AICAR or its derivatives are the prototypes of a new family of compounds for the treatment of hyperglycemia and insulin resistance characteristic of type 2 diabetes and can help in maintaining glucose homeostasis, particularly when other treatments have failed or are no longer successful.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A method for prophylaxis or treatment of a disease or condition associated with hyperglycemia, insulin resistance, insulin resistance syndrome or obesity, said method comprising the steps of:
providing a patient suffering from or believed to be at risk of suffering from a disease or condition associated with hyperglycemia, insulin resistance, insulin resistance syndrome or obesity; and
administering to said patient a therapeutic composition comprising an effective amount of an activator of AMP-activated protein kinase (AMPK), wherein said AMPK activator furthermore is effective at decreasing malonyl coenzyme A (malonyl CoA) levels in said patient and wherein said composition is administered to said patient over a period of time of at least 28 days.

2. The method of claim 1, wherein said activator in said composition is administered to said patient at a dosage of 5 mg/kg/day to 100 mg/kg/day.

3. The method of claim 1, wherein administration of said composition to said patient over said period of time occurs intermittently and no more frequently than every other day.

4. The method of claim 3, wherein said intermittent administration is every other day.

5. The method of claim 3, wherein said intermittent administration is twice a week.

6. The method of claim 3, wherein said intermittent administration is once a week.

7. The method of claim 1, wherein administration of said composition to said patient is subcutaneous.

8. The method of claim 1, wherein administration of said composition to said patient is oral.

9. The method of claim 1, wherein said therapeutic composition comprises 5-aminoimidazole-4-carboxamide-1-β-D-ribofuranoside (AICAR).

10. The method of claim 1, wherein said therapeutic composition comprises an analog of AICAR.

11. The method of claim 1, wherein said therapeutic composition comprises a precursor of AICAR.

12. The method of claim 1, wherein said therapeutic composition is administered to said patient for at least one month.

13. The method of claim 1, wherein said therapeutic composition is administered to said patient for at least three months.

14. The method of claim 1, wherein said disease or condition is vascular disease associated with metabolic abnormalities

15. The method of claim 143, wherein said disease or condition is atherosclerotic vascular disease.

16. The method of claim 14, wherein said disease or condition is selected from the group consisting of blindness, retinopathy and nephropathy caused by diabetic microvascular disease.

17. A method of identifying an agent for prophylaxis or treatment of a disease or condition associated with hyperglycemia, insulin resistance, insulin resistance syndrome or obesity, said method comprising the steps of:
(a) providing a model system for mammalian intracellular metabolism;
(b) testing a candidate agent for an ability to activate AMPK in said model system;
(c) selecting a candidate agent identified in step (b) as having an ability to activate AMPK in said system;
(d) testing said agent identified in step (c) for an ability furthermore to decrease malonyl CoA levels in said system; and
(e) selecting a candidate agent identified in step (d) as furthermore having an ability to decrease malonyl CoA levels in said system as an agent for prophylaxis or treatment of said disease or condition.

18. An agent for prophylaxis or treatment of a disease or condition associated with hyperglycemia, insulin resistance, insulin resistance syndrome or obesity identified by the method of claim 17.

19. A method of identifying an agent for prophylaxis or treatment of a disease or condition associated with hyperglycemia, insulin resistance, insulin resistance syndrome or obesity, said method comprising the steps of:
(a) providing a model system for mammalian intracellular metabolism;
(b) testing a candidate agent for an ability to block activation of a capace in said model system; and
(c) selecting a candidate agent identified in step (b) a having an ability to block activation of a capace in said model system as an agent for prophylaxis or treatment of said disease or condition.

20. An agent for prophylaxis or treatment of a disease or condition associated with hyperglycemia, insulin resistance, insulin resistance syndrome or obesity identified by the method of claim 19.
